(19) United States
(12) Patent Application Publication
(10) Pub. No.: US 2009/0136052 A1
(43) Pub. Date: May 28, 2009

(54) Active Noise Cancellation Using a Predictive Approach

(75) Inventors: Robert G. Hohlfeld, Attleboro, MA (US); Allan E. Sheppard, JR., Holden, MA (US)

Correspondence Address: MILLS & ONELLO LLP, ELEVEN BEACON STREET, SUITE 605, BOSTON, MA, 02108, US

(73) Assignee: DAVID CLARK COMPANY INCORPORATED, Worcester, MA (US)

(21) Appl. No.: 11/945,494

(22) Filed: Nov. 27, 2007

(51) Int. Cl. A61F 11/06

(52) U.S. Cl. 381/71.1

(57) ABSTRACT

A method for active noise cancellation in a volume generates a mathematical model of a noise process to be cancelled. Using the mathematical model, a noise signal in a next sample period is predicted from a measured noise signal. The predicted signal is inverted and applied to the volume. Destructive interference of the noise signal and the inverted signal cancels the noise in the volume.

### BACKGROUND OF THE INVENTION

1. Field of the Invention

The invention relates generally to active cancellation of acoustic noise, and relates in particular to active noise cancellation by the introduction of a signal canceling said noise by destructive interference within a volume or a headset, with the canceling signal computed digitally on the basis of a mathematical model of the noise to be cancelled.

2. Description of the Related Art

Active noise control and cancellation is of obvious importance for situations in which human beings must operate in an environment with high noise levels. Noise control and cancellation is often required to prevent injury to the human auditory system by high ambient noise levels. Mental processes of humans under these conditions are often impaired, to say nothing of discomfort to humans exposed to high levels of acoustic noise.

Active noise cancellation (or control) works on the principles of destructive interference of acoustic waves, a principle well understood in physical acoustics. In the volume of interest, for example, the interior of a headset dome or any other volume in which noise levels are being controlled, there will be a sound pressure wave fluctuating as some function of time, p(t). Noise cancellation operates by the determination of an “anti-noise” signal, which is the additive inverse of this noise signal, −p(t), and introducing this signal into the volume to be noise-controlled, usually by means of a loudspeaker. In the volume to be noise-controlled, the noise signal and the anti-noise signal destructively interfere and cancel out, leaving a much reduced noise level within the noise-controlled volume. The level to which noise in the noise-controlled volume is reduced will usually depend on the accuracy in amplitude and time to which the anti-noise signal is determined and the fidelity with which this determined signal is projected acoustically into the noise-controlled volume. The principal issue distinguishing different approaches to active noise cancellation is the means by which the canceling anti-noise signal is generated before it is introduced into the noise-controlled volume of interest.

In conventional implementations of active noise cancellation, the anti-noise signal is generated as a result of feedback and/or feedforward processes implemented by a variety of analog and/or digital circuitry means. Different inventions in these fields are distinguished by the means by which these processes are affected by these analog and/or digital circuitry means.

In the conventional implementations, feedback involves measuring the sound level in the headset dome (or other volume to which active noise control is to be applied) and generating the cancellation signal to be applied to the headset speakers according to the criterion that the net noise level, noise signal plus anti-noise level, is minimized. Feedforward, on the other hand, involves measuring the noise level at some point exterior to the headset dome and then using a transfer function, which is a model for how noise propagates into the headset dome, to generate a cancellation signal which is then applied to the headset speakers. Some noise-canceling headsets use a combination of feedback and feedforward techniques.

Almost all of the conventional active noise-canceling headsets use analog circuitry to generate feedback and/or feedforward signals to be used for the noise cancellation. Among the advantages of using analog circuitry for these problems is its speed of operation as compared to digital circuitry, which is explained below, and its similarity to other kinds of well-established audio circuitry technology. Nonetheless, there is considerable interest in developing corresponding noise cancellation technology using digital circuitry. The advantages of a digital noise canceling system include (but are not limited to) more flexible implementations, that can be updated simply by loading new software or firmware in a system, and improved quality and cost control in manufacture.

Difficulties arise in practice with digital noise canceling systems because they often reduce in practice to a, usually adaptive, Finite Impulse Response (FIR) filter operation, or its equivalent in operations generating a digital feedback signal. To cover a useful frequency range for an application in, for example, civil aviation, the frequency of operation must extend from a range of a few kHz at the high frequency range of operation down to a small number of tens of Hz on the low frequency range of operation. This corresponds to a FIR with many tens to a few hundred taps, alternatively, digital filter coefficients. This has some undesirable consequences. First, the computational load is very high, putting the digital approach in the domain of very high-end digital signal processing (DSP) chips, or out of the range of presently available DSP chips altogether. In addition, the large number of taps imposes a long filter delay, which means that the computed anti-noise signal lags behind the noise signal, causing incomplete cancellation, particularly at high frequencies. The long filter delay causes difficulties when the noise is time-varying on time scales comparable to the filter delay. Further, the noise cancellation performance can be highly frequency dependent, for reasons elaborated upon below.

Some of the conventional implementations have tried to implement active noise cancellation approaches based on the class of least-mean-squares (LMS) algorithms. This is a conventional choice based on the desire to reduce the noise power (squared amplitude of the pressure) in the noise-canceling volume of interest. These approaches are generally reducible to the well-understood approach of Wiener filtering. These approaches share the limitation of the digital approaches discussed above and have the additional characteristic of being very much more effective for a “tonal” noise signal than for a noise signal which is better described as broadband noise. A “tonal” noise signal in this context refers to noise or components of the noise which may be well-characterized by a relatively small number of sinusoidal coefficients.

### SUMMARY OF THE INVENTION

One feature and advantage of the present invention is to provide an active noise cancellation method by which a very high degree of noise cancellation can be achieved for headset applications approaching a level of performance limited by bone conduction through the human head.

Another feature and advantage of the present invention is to provide an active noise cancellation method based on extrapolation of a mathematical model of the noise process being cancelled, thus avoiding the pitfalls of methods based on conventional feedback and feedforward processes.

Another feature and advantage of the present invention is to provide an active noise cancellation method using mathematical models, such as a Maximum Entropy Method, well-suited to extrapolation-based noise cancellation due to avoiding divergences in the extrapolation process.

Another feature and advantage of the present invention is to provide an active noise cancellation method well-suited to operation over a wide range of audible frequencies by avoiding construction of digital FIR filters with a very large number of taps with corresponding filter delays and large computational loads.

Another feature and advantage of the present invention is to provide an active noise cancellation method which is very computationally efficient and thus suited to implementation on DSP chips.

Another feature and advantage of the present invention is to provide an active noise cancellation method that can treat tonal and non-tonal noise components on an equal basis, thus not modifying the perceptual character of the residual noise field after cancellation. This may be important for the user to properly evaluate operation of machinery or hazards in his or her environment.

Further features and advantages are to provide a highly effective active noise cancellation method which is suitable for implementation in digital hardware and thus has important advantages in ease and reproducibility of manufacture. Still further features and advantages will become apparent from a consideration of the ensuing description and drawings.

The present invention is directed to a method of active noise cancellation in a volume that includes generating a mathematical model of a noise process to be cancelled and constructing an extrapolated noise signal from the mathematical model. The method further includes inverting the extrapolated signal, applying the inverted signal to the volume, and canceling the noise in the volume using the applied signal.

In one embodiment, the method includes measuring the noise signal close to the volume, converting the noise signal to a digital signal, and inputting the noise signal to the mathematical model.

In one embodiment, the method includes, before applying the inverted signal to the volume, converting the inverted signal into an analog signal.

In one embodiment, the method includes generating an anti-noise sound wave by a speaker operating within the volume. In another embodiment, destructive interference of the noise signal and the anti-noise sound wave cancels the noise.

In one embodiment, the mathematical model is generated using at least one of linear predictive coding and maximum entropy method.

In one embodiment, the mathematical model is used to predict the noise signal in the next sample. In another embodiment, the predicted signal for the next sample is used to cancel the noise signal in the next sample.

In one embodiment, the mathematical model is periodically updated.

In one embodiment, canceling the noise includes canceling tonal and non-tonal noise components.

In one embodiment, the method includes measuring the noise outside the volume.

In one embodiment, the method includes measuring the noise outside the volume and measuring the noise within the volume.

In one embodiment, canceling the noise comprises canceling periodic noise and noise generated by turbulent air flow.

In one embodiment, the volume comprises at least one of a helmet, a headset, and speakers close to a head.

In accordance with another aspect of the invention, the invention is directed to a method of active noise cancellation in a volume that includes measuring a noise signal and predicting a noise signal in a next sample period from the measured noise signal. The method further includes inverting the predicted signal, applying the inverted signal to the volume, and canceling the noise signal in the next sample period using the predicted signal.

In one embodiment, the method includes, prior to predicting the noise signal, converting the noise signal to a digital signal.

In one embodiment, the method includes, before applying the inverted signal to the volume, converting the inverted signal into an analog signal.

In one embodiment, the method includes generating an anti-noise sound wave by a speaker operating within the volume. In another embodiment, destructive interference of the noise signal and the anti-noise sound wave cancels the noise.

In one embodiment, the predicted noise signal is generated using at least one of linear predictive coding and a maximum entropy method.

In one embodiment, canceling the noise signal includes canceling tonal and non-tonal noise components.

In one embodiment, the method includes measuring the noise outside the volume.

In one embodiment, the method includes measuring the noise outside the volume and measuring the noise within the volume.

In one embodiment, the canceling the noise signal comprises canceling periodic noise and noise generated by turbulent air flow.

In one embodiment, the volume comprises at least one of a helmet, a headset, and speakers close to a head.

In accordance with another aspect of the invention, the invention is directed to an apparatus for active noise cancellation in a volume that includes a first microphone that senses noise and a noise model generator that receives the sensed noise and generates a mathematical model of a noise process to be cancelled. The apparatus further includes an extrapolator that extrapolates a noise signal from the mathematical model and an inverter that inverts the extrapolated signal and applies the signal to a volume.

In one embodiment, the apparatus includes an analog-to-digital converter that receives the sensed noise signal from the first microphone and applies a digitized representation of an analog signal to the noise model generator.

In one embodiment, the apparatus includes a digital-to-analog converter that receives the inverted signal from the inverter and applies an analog equivalent of the digital signal to the volume.

In one embodiment, the first microphone senses the noise outside the volume.

In one embodiment, the apparatus includes a second microphone. In another embodiment, the second microphone senses the noise signal within the volume.

In one embodiment, the noise model generator comprises at least one of a digital signal processor, a computer and a field-programmable gate array.

In one embodiment, the noise model generator uses at least one of linear predictive coding and a maximum entropy method.

In one embodiment, the noise model generator calculates an extrapolated value of the noise signal for a next sample period.

In one embodiment, the extrapolator constructs the extrapolated noise signal for a future sample period.

In one embodiment, the mathematical model is periodically updated.

In one embodiment, the apparatus cancels tonal and non-tonal noise components.

In one embodiment, the apparatus cancels periodic noise and noise generated by turbulent air flow.

In one embodiment, the volume comprises at least one of a helmet, a headset, and speakers close to a head.

In accordance with another aspect of the invention, the invention is directed to an apparatus for active noise cancellation in a volume that includes a first microphone that senses a noise signal and a noise predictor that predicts a noise signal in a next sample period from the sensed noise signal. The apparatus further includes an extrapolator that extrapolates a noise signal from the predicted noise signal and an inverter that inverts the extrapolated signal and applies the inverted signal to the volume.

In one embodiment, the apparatus includes an analog-to-digital converter that receives the sensed noise signal from the first microphone and applies a digitized representation of an analog signal to the noise predictor.

In one embodiment, the apparatus includes a digital-to-analog converter that receives the inverted signal from the inverter and applies an analog equivalent of the digital signal to the volume.

In one embodiment, the first microphone senses the noise outside the volume.

In one embodiment, the apparatus includes a second microphone. In another embodiment, the second microphone senses the noise signal within the volume.

In one embodiment, the noise predictor comprises at least one of a digital signal processor, a computer and a field-programmable gate array.

In one embodiment, the noise predictor uses at least one of linear predictive coding and maximum entropy method.

In one embodiment, the noise predictor calculates an extrapolated value of the noise signal for a next sample period.

In one embodiment, the extrapolator constructs the extrapolated noise signal for a signal for a next sample period.

In one embodiment, the predicted noise signal is periodically updated.

In one embodiment, the apparatus cancels tonal and non-tonal noise components.

In one embodiment, the apparatus cancels periodic noise and noise generated by turbulent air flow.

In one embodiment, the volume comprises at least one of a helmet, a headset, and speakers close to a head.

### BRIEF DESCRIPTION OF THE DRAWINGS

The foregoing and other objects, features and advantages of the invention will be apparent from the more particular description of preferred aspects of the invention, as illustrated in the accompanying drawings in which like reference characters refer to the same parts throughout the different views. The drawings are not necessarily to scale, emphasis instead being placed upon illustrating the principles of the invention.

FIG. 1 is a schematic functional block diagram of a model-based active noise cancellation system in accordance with an embodiment of the invention.

FIG. 2 is a schematic functional block diagram of an alternative model-based active noise cancellation system.

FIG. 3 is a schematic functional block diagram of a hardware system implementing the functional block representation of the system illustrated in FIG. 2.

FIG. 4 is a logical flow diagram illustrating operation of the invention when extrapolated values and updates to a predictive noise model are generated in synchronism with a sampling period of data collection in accordance with an embodiment of the invention.

FIG. 5 is a logical flow diagram illustrating the operation of the invention when the predictive noise model is updated less frequently than the data collection sampling period and the output period of anti-noise to the noise cancellation hardware in accordance with an embodiment of the invention.

FIG. 6 illustrates a schematic of noise produced by a single turbulent element of air flow passing past the active noise cancellation system of FIGS. 1, 2 and 3.

FIG. 7 illustrates a relation between a noise signal, a MEM extrapolated signal, and a residual signal.

FIGS. 8A, 8B and 8C illustrate a segment of aircraft noise data and matching segments of noise cancelled data after application of the MEM noise cancellation approach of the invention.

### DETAILED DESCRIPTION OF PREFERRED EMBODIMENTS OF THE INVENTION

The present invention is particularly shown and described with reference to noise control in aviation headsets, an area of great importance in active noise cancellation applications, but it will be understood by those of ordinary skill in the art that the principles described here are also applicable to active noise control in other areas, including the control of noise in a region of free space not bounded by physical structures such as the walls of a headset dome. The active noise cancellation can be applied to headsets, helmets and instances where speakers are close to a head.

In the present invention, the general approach is to construct a mathematical model of the noise process to be cancelled out and to extrapolate that signal forward in time. The extrapolated signal is inverted (multiplied by −1) and applied to loudspeakers, or other sound production means, to cancel out the noise signal in the volume in which active noise cancellation is desired. The mathematical model of the noise process must be updated at various times so as to remain a good representation of the noise process for extrapolation purposes.

Many conventional mathematical models, such as those based on polynomial extrapolation, are unsuitable models for a physical system such as this because of the well-known property of polynomial extrapolation to diverge as the extrapolation range increases. Other conventional mathematical models based on Fourier analysis are also problematic because extrapolation of sinusoidal models of the noise cannot capture the stochastic evolution of the sound field in many important applications, such as cancellation of the noise encountered in civil aviation. Furthermore, the physical characteristics of the noise process to be cancelled must be taken into account in determining the appropriate mathematical model.

In the preferred embodiment, a Maximum Entropy Method (MEM) is used as the basis of a noise cancellation mathematical model. MEM was originally applied to problems in spectral analysis and now has its broadest applications in problems of image restoration.

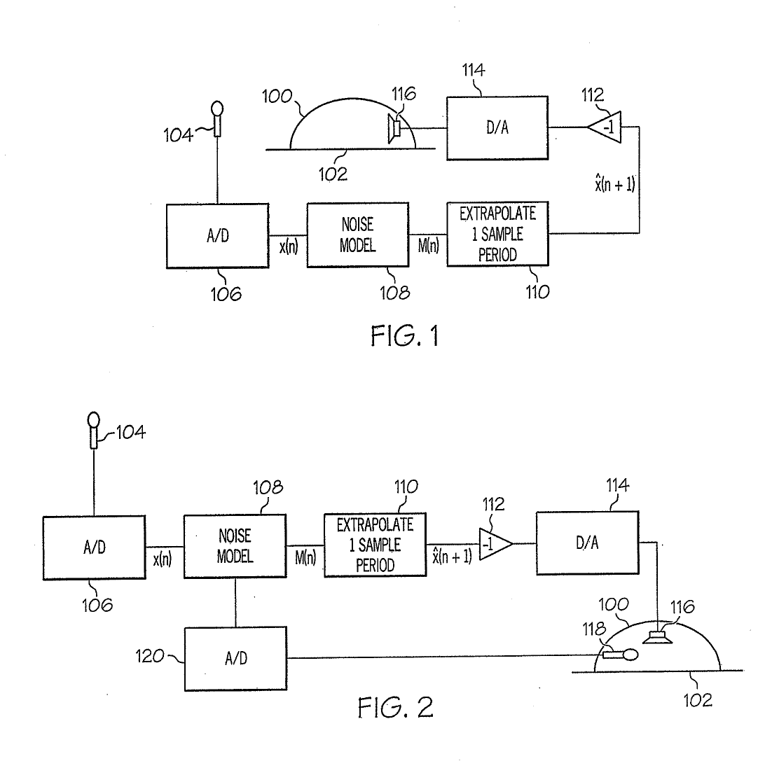

FIG. 1 is a schematic functional block diagram of a model-based active noise cancellation system in accordance with an embodiment of the invention. The system is a noise-canceling headset, although the principals illustrated here can be applied to noise-cancellation in other circumstances. The volume in which noise is to be cancelled is bounded by a headset dome 100, which is resting on a head 102. An ambient noise field to be cancelled is measured by a microphone 104. A practical noise-canceling system will have the microphone situated physically close to the headset dome 100. The analog noise signal measured by the microphone 104 is converted to a digital signal x(n) by an analog-to-digital converter (A/D converter) 106. The digital signal x(n) is the binary representation of the noise signal at time step n. The digital signal x(n) is an input to a noise model generator 108 which is implemented in a digital signal processor, general purpose computer, or field-programmable gate array (FPGA). In the preferred embodiment, as elaborated upon below, the mathematical noise model will be based on the MEM. The digital signal processor or general purpose computer is used to calculate an extrapolated value of the noise signal one sample period later than the measured value. Construction of the extrapolated noise signal \hat{x}(n+1) occurs in the extrapolator 110 which extrapolates a noise signal from the mathematical model. Due to an “anti-noise” signal being required for noise-cancellation, the extrapolated noise signal is multiplied by −1 in a digital signal inverter 112. The signal inversion is illustrated as being performed digitally, although it could also be performed in hardware in a slightly modified embodiment. Finally, the digital anti-noise signal is converted back to the analog domain in a digital-to-analog converter (D/A converter) 114. The analog anti-noise signal is used to generate a physical anti-noise sound wave by a speaker 116 operating within the headset dome 100. Destructive interference of the noise sound wave and the anti-noise sound wave leads to the desired noise cancellation.

The system, as illustrated in FIG. 1, is designed to operate in such a fashion that the timing of the output anti-noise sound wave occurs at a physical time corresponding to the extrapolated noise model time of n+1. At which time, a fresh noise measurement has been taken by the microphone and is already in the processing pipeline. Rather than a processing cycle of duration of one sample period, a longer duration processing cycle could be adopted in conjunction with an extrapolation of the noise model for the corresponding number of sample periods.

FIG. 2 is a schematic functional block diagram of an alternative model-based active noise cancellation system. FIG. 2 illustrates an elaboration to the embodiment shown in FIG. 1. In FIG. 2, a second microphone 118 measures a cancelled noise field within the headset dome 100 so that a second analog-to-digital converter 120 can provide a digital measurement of the actual noise level achieved within the headset dome 100 to be used in the mathematical noise model generator 108. In the operation of the embodiment shown in FIG. 1, there is no guarantee that the overall digital gain implicit in the mathematical model and the physical gain associated with analog components such as amplifiers and the speaker is set to an overall unity gain accurately enough to yield accurate noise cancellation. Furthermore, because the noise measurement microphone 104 is outside the headset dome 100, and the volume for which noise cancellation is to be effected is within the headset dome 100, the mathematical noise model must include some representation of the acoustic transfer function of the headset dome 100 for the ambient noise signal.

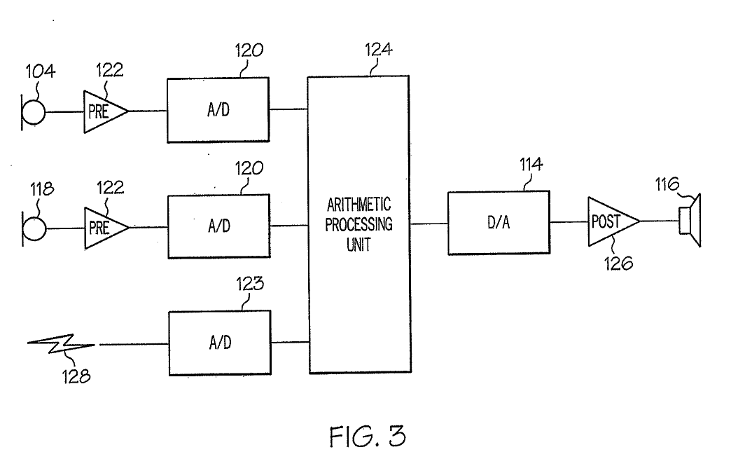

FIG. 3 is a schematic diagram of hardware implementation of the noise-canceling headset system according to an embodiment of the invention. For clarity, a single channel is shown that would control the noise level inside a single headset dome 100. Two microphones 104 and 118 are illustrated, one of which, microphone 104, would be deployed just outside the headset dome 100, to measure the noise field to be cancelled. The second microphone 118 may be deployed within the headset dome 100, to support a second calculation or to provide other information as dictated by the details of a particular embodiment. Each microphone has a pre-processing amplifier 122, that pre-conditions the signal and compensates for differences in the efficiency of different microphones. The signal(s) from the pre-processing amplifiers 122 are converted to digital signals in A/D converters 106 and 120 and the digital values are passed on to an arithmetic processing unit 124. The arithmetic processing unit 124 corresponds to the mathematical noise model generator 108, extrapolator 110 and inverter 112 of FIG. 2. The arithmetic processing unit 124 uses the digitized noise field data as inputs into the MEM model (or another extrapolative model within the scope of this invention), and constructs extrapolation values of the noise field, phase inverted to cancel out the noise in the control volume. The arithmetic processing unit can be a general purpose computer, a digital signal processing chip, a field-programmable gate array, or any other digital data processing means that carries out the mathematical model construction and, from the mathematical model, generates the values of the phase-inverted signal. The digital noise cancellation signal is fed into a D/A converter 114, and the resulting analog signal is fed to a post-processing amplifier 126. The post-processing amplifier 126 drives a speaker 116 deployed within the headset dome 100, or other volume in which the noise is to be canceled. The post-processing amplifier 126 compensates for different efficiencies of the speaker 116 from unit to unit.

A communications channel 128 is shown as an input to the arithmetic processing unit 124 so that critical communications, such as radio communications with a tower or air traffic controller, or entertainment signals, can be passed on to the user through the headset. The communications channel 128 is converted to a digital signal in A/D converter 123 and the digital values are passed on to an arithmetic processing unit 124. The signals would be retained at a desired signal level after the objectionable aircraft noise has been cancelled out.

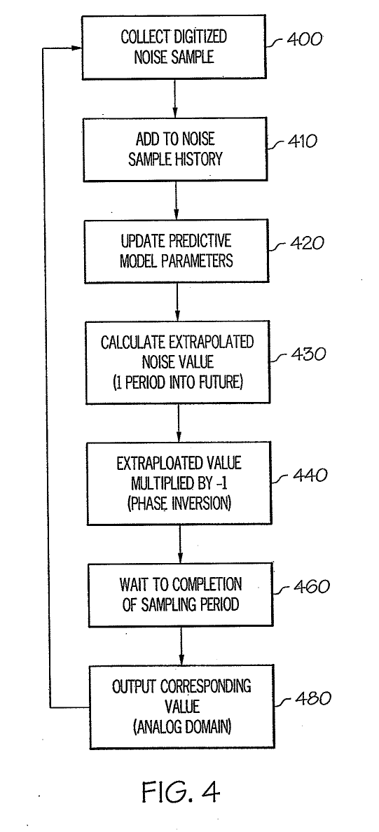

FIG. 4 is a logical flow diagram illustrating operation of the invention when extrapolated values and updates to the predictive noise model are generated in synchronism with the sampling period of data collection. FIG. 4 is a logical flow diagram illustrating the connections between computational operations carried out in the preferred embodiment of this invention. In this embodiment, the predictive model operates once per data sampling period. The predictive model needs only to compute an extrapolated noise sample one sampling period into the future, which can usually be done with relatively high accuracy provided that the sampling period is not too short and the computational platform is sufficiently powerful to complete the update of model parameters and compute the extrapolated noise value for output in the analog domain. In FIG. 4, in step 400, a digitized noise sample is collected. In step 410 of FIG. 4, the noise sample is added to the noise sample history. In step 420 of FIG. 4, the predictive model parameters are updated. In step 430 of FIG. 4, an extrapolated noise value that is one period into the future is calculated using the predictive model. In step 440 of FIG. 4, the extrapolated value is phase inverted by multiplying the extrapolated value by -1. Then in step 460 of FIG. 4, the process waits until the completion of a sampling period. Then in step 480 of FIG. 4, the corresponding noise value is output in the analog domain. Then the process returns to the first step 400 of collecting a digitized noise sample.

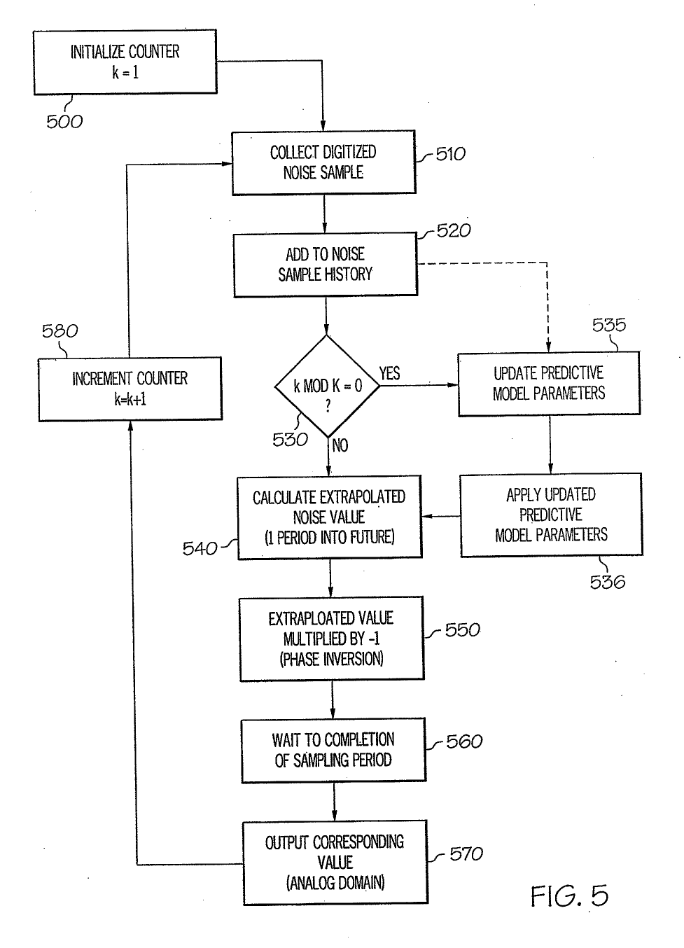

FIG. 5 is a logical flow diagram illustrating the operation of the invention when the predictive noise model is updated less frequently than the data collection sampling period and the output period of anti-noise to the noise cancellation hardware. FIG. 5 is a logical flow diagram disclosing another embodiment of the invention. In this embodiment the model is updated using data collected at the hardware sampling rate, but the predictive model, as applied to the computation of extrapolated noise values is updated only once every K samples, that is a total of K samples are extrapolated and output using a single set of model parameters in the predictive model. After K sampling periods have elapsed, the model parameters are updated and the updated predictive model is used to generate the next K extrapolated noise values. This coordination of sample collection and model update may be carried out in a variety of ways, but for purposes of illustration, it is shown in FIG. 5 using a counter incremented as each new sample is collected and modular arithmetic to control the update of the predictive model. An approach such as is illustrated in FIG. 5 would be attractive when computational power on available hardware is limited and the number of computational operations required to evaluate a new set of model parameters is very much larger than the number of computational operations necessary to compute new extrapolated noise values. In step 500 of FIG. 5, the counter is initialized to k=1. In step 510 of FIG. 5, a digitized noise sample is collected. In step 520 of FIG. 5, the digitized noise sample is added to the noise sample history. Data flows into step 535 as it is gathered, that is in each sampling period, so that 535 has K samples when it needs to update the model, i.e., when it is triggered by step 530. In step 530 of FIG. 5, using modular arithmetic it is determined whether K samples have been taken. If K samples have not been taken, the process proceeds to step 540 in which an extrapolated noise value is calculated one period into the future. If K samples have been taken then the process proceeds to step 535. In step 535 of FIG. 5, the predictive model parameters are updated. Then, in step 536 of FIG. 5, the updated predictive model parameters are applied and the process proceeds to step 540 in which an extrapolated noise value is calculated one period into the future. Following step 540 of FIG. 5, in step 550, the extrapolated value is phase inverted by multiplying the extrapolated value by −1. The process, in step 560 of FIG. 5, waits until the completion of a sampling period and when the sampling period is completed, in step 570 of FIG. 5, the corresponding noise value is output in the analog domain. The process increments the counter to k=k+1, in step 580, and returns to the first step 510 of collecting a digitized noise sample.

For the issue of making a noise-canceling headset for aviation applications, the noise is composed of a combination of periodic noise generated by the aircraft engine and propeller, including turbines, turbofans and associated transmissions, as well as the turbulent flow around the aircraft cabin. The periodic noise generated by aircraft machinery is readily predictable due to its periodicity, even if it is not sinusoidal. The noise generated around the aircraft by turbulent air flow is more problematic.

The turbulent boundary layer is viewed as being composed of a set of “eddies” or “vortices” in the flow that have some characteristic “overturning frequency”. A component of the noise in the aircraft cabin corresponds to this frequency. The amplitude of the vortices grows and decays and in many turbulent flows there is a strong intermittency in the flow field. Individual vortices are carried past the cabin by the aircraft motion and so their contribution necessarily grows and decays for that reason as well. The noise field in the aircraft cabin due to turbulent flow around the cabin will be made up of the incoherent sum of a large number of these components, each with differing frequencies and amplitudes, growing and decaying in time. Over time scales of a tenth of a second, approximately, and longer, the impression to the human ear is that of a random white noise signal. Aircraft noise has predictable components when superimposed over tenths of milliseconds.

The implication of the above paragraph is that any attempt to build a predictive model of the noise in an aircraft cabin over periods greater than tenths of milliseconds is difficult. Furthermore, techniques as implemented in the present state of the art that have digital filters with very long filter delays, again in the range of tenths of milliseconds or longer, will similarly experience difficulties in matching the random variability of the noise field on longer time scales. On the other hand, an approach taking advantage of modern digital electronics and processors to operate on much shorter time scales can be effective. On time scales of a few microseconds, the noise field around a headset dome will be composed of engine (including turbine engines) and propeller noise (tonal components and hence highly predictable) and a small number of noise signals arising from turbulent cells passing by the aircraft cabin close by. These latter signals are also relatively predictable for the limited time before they decay away and can thus be modeled and phase-cancelled away. The turbulent noise elements are replaced over a time scale of tenths of milliseconds with statistically independent noise elements and owing to their statistical independence a model predicting their noise behavior must be different from a noise model operating a few tenths of milliseconds earlier to cancel out the earlier turbulent noise elements. Again, relative predictability on short time scales is replaced by fundamental unpredictability on longer time scales.

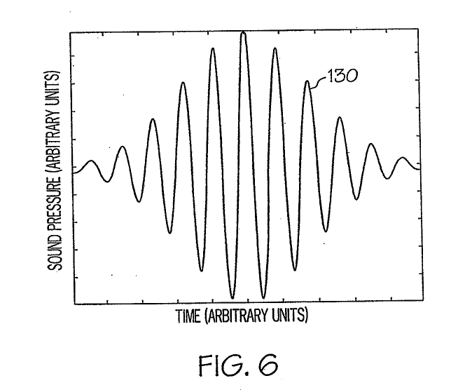

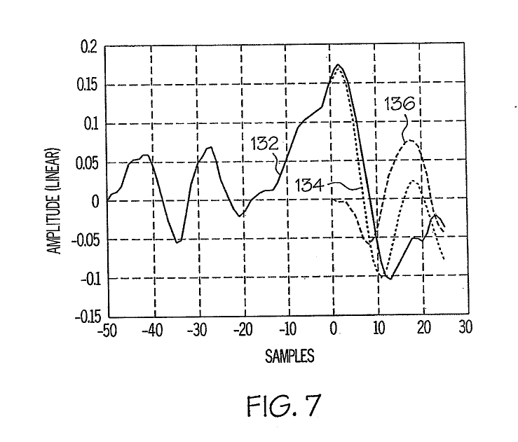

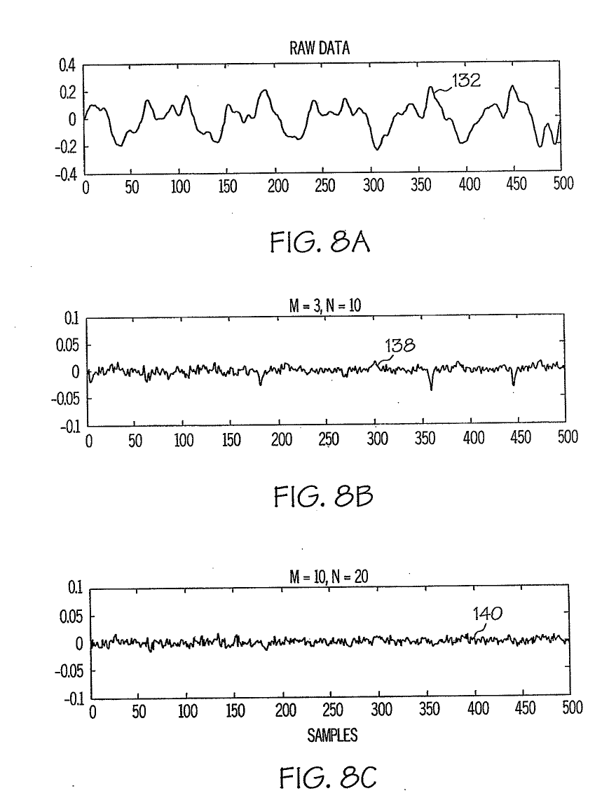

FIG. 6 illustrates a schematic of noise produced by a single turbulent element of air flow passing past the active noise cancellation system of FIGS. 1, 2 and 3. FIG. 6 is a schematic noise trace generated by a single turbule 130 which illustrates a sinusoidal characteristic frequency with an envelope that is growing and decaying in time. A mathematical model suitable for noise cancellation must be able to simultaneously cope with periodic noise signals and the incoherent sum of many signals as illustrated in FIG. 6, with different frequencies and times of maximum signal amplitude.

FIG. 7 illustrates a relation between a noise signal, a MEM extrapolated signal, and a residual signal. FIG. 7 shows the operation of noise cancellation using a mathematical predictive approach acting on a recorded aircraft noise signal 132 (solid line). The time axis is in units of the sampling period and the prediction of the noise signal begins at sample number 0. Negative sample numbers denote past sample values used to construct the MEM model. Positive sample numbers represent times for data during the extrapolation period. The extrapolated signal 134 (dashed line) is based on a mathematical model constructed using data up to sample 0, and is shown extrapolated to times greater than sample 0. The residual signal 136 (dotted line) is the recorded aircraft noise signal minus the extrapolated signal and would be the sound wave signal inside the headset dome 100 after the speaker radiates the negative of the extrapolated signal in that controlled volume for operation of the noise cancellation.

It should be noted that the extrapolated signal is a good representation of the recorded aircraft noise for a small number of samples of extrapolated time, i.e., for a small number of samples after sample 0, as shown by the small amplitude of the residual signal in that range. The residual signal grows in amplitude as more time elapses after sample 0 and so the accuracy of the extrapolated signal in representing the recorded aircraft noise deteriorates markedly as the extrapolation is extended in time.

FIGS. 8A, 8B and 8C show a segment of 500 samples of recorded aircraft cabin noise at a sampling rate of 11025 samples per second with successively higher order MEM noise cancellation models applied. The first trace 132, illustrated in FIG. 8A, is recorded aircraft noise with no MEM noise cancellation applied. The second trace 138, in FIG. 8B, is the cancelled noise after an MEM model using three MEM coefficients derived from processing the most recent ten terms in the single sample projection operation illustrated in FIG. 4. Significant reduction of the noise amplitude is evident. The third trace 140, in FIG. 8C, is the cancelled noise remaining after an MEM model using ten MEM coefficients derived from processing the most recent twenty terms in the single sample projection operation shown in FIG. 4. Still, more reduction in the residual noise amplitude can be seen. It should be noted that the amplitude scale of the second trace 138 and the third trace 140 are significantly expanded compared to the raw data in the first trace 132, owing to the much reduced amplitude after operation of the noise cancellation.

The details of constructing the correct mathematical model to be implemented in a computational platform, i.e. a digital signal processor chip, general-purpose computer, or field-programmable gate array, will now be described. In a preferred embodiment, a form of linear predictive coding known as the Maximum Entropy Method (MEM) that has a number of desirable advantages is used.

It is assumed that there is a set of measured values {y_{α}′} corresponding to the true values of some physical process y, denoted by {y_{α}}. The measured values are related to the true values by the addition of random noise,

y₆₀′=y_{α}+n_{αa} (1)

Some care in interpretation is in order here regarding the meaning of “noise.” The acoustic noise in an aircraft cabin, while it is “noise” in conventional human terms, is in this case the signal of interest. The mathematical noise, n_{α}, represents such effects as quantization noise in the digital system and electronic noise in amplifiers, microphones, speakers and other system components.

The objective is to obtain the best estimator of the true value of a particular data point, y*, based on a linear combination of known, noisy values. Recall that known, noisy values are the only quantities known in practice. The construction of y* thus takes the form

$${y}_{*} = {\sum }_{α} {d}_{* α} {y}_{α}^{′} + {x}_{*} , \qquad ( 2 )$$

where the coefficients d*_{α} that minimize, according to some practical figure of merit (usually rms power), the discrepancy x*. The estimation coefficients d*_{α} have a “star” subscript to indicate that they depend on the choice of the point y*. For the problem of interest, the determination of y* as an extrapolated signal value is properly determined as one of linear prediction or as an application of the MEM.

If minimizing the discrepancy x* in the statistical mean square sense (which will have the effect of minimizing the power of the noise-cancelled pressure wave in the headset dome 100) is chosen, the equation can be written

$$\begin{matrix}〈 {x}_{*}^{2} 〉 = 〈 {[ {\sum }_{α} {d}_{{*}_{α}} ( {y}_{α} + {n}_{α} ) - {y}_{*} ]}^{2} 〉 \\ = {\sum }_{α , β} ( 〈 {y}_{α} {y}_{β} 〉 + 〈 {n}_{α} {n}_{β} 〉 ) {d}_{* α} {d}_{* β} - 2 {\sum }_{α} 〈 {y}_{*} {y}_{α} 〉 {d}_{* α} + 〈 {y}_{*}^{2} 〉\end{matrix} \qquad ( 3 )$$

as an equation for linear prediction (MEM) coefficients, the d*_{α}'s, that minimize the mean square discrepancy. Here angle brackets denote a statistical average.

Equation (3) makes the usual assumptions that the noise and signal are uncorrelated so that [symbol] n_{α}y_{β} [symbol] =0, together with similar cross products. The quantity [symbol] y_{α}y_{β} [symbol] is the autocorrelation function of the signal, while [symbol] n_{α}n_{β} [symbol] represents the autocorrelation of the noise. For the important practical special case in which the noise is uncorrelated point-to-point, [symbol] n_{α}n_{β} [symbol] = [symbol] n_{α}² [symbol] δ_{αβ}, with δ_{αβ} the Kronecker delta function. It is conventional to define the correlation quantities as matrices and vectors,

φ_{αβ}≡ [symbol] y_{α}y_{β} [symbol] φ*_{α}≡ [symbol] y*y_{α} [symbol] η_{αβ}≡ [symbol] n_{α}n_{β} [symbol] or [symbol] n_{α}² [symbol] δ_{αβ}. (4)

Setting the derivative of Equation (3) with respect to the d*_{α}'s equal to zero, one obtains a set of linear equations in terms of the linear prediction coefficients,

$${\sum }_{β} [ {φ}_{αβ} + {η}_{αβ} ] {d}_{* β} = {φ}_{* α} . \qquad ( 5 )$$

If the solution is written as a matrix inverse, the estimation equation, Equation (2) becomes (omitting the minimized discrepancy x*),

$${y}_{*} \approx {\sum }_{α , β} {{φ}_{* α} [ {φ}_{μ v} + {η}_{μ v} ]}_{αβ}^{- 1} {y}_{β}^{t} . \qquad ( 6 )$$

This formalism can be extended specifically to the case where data points equally spaced in time, yᵢ, i=1,2,K,N are given and M consecutive values of yᵢ to predict the M+1st are used. It is assumed that the noise and the signal being predicted are stationary over the interval of M time steps. The assumption of the noise and the signal being predicted are stationary guarantees that the autocorrelation [symbol] yⱼyₖ [symbol] depends only on the difference |j−k|, that is on the lag in samples in the computation of the autocorrelation function. Given that the autocorrelation is of this form, it can be represented as a function of a single index,

$${φ}_{j} ≡ 〈 {y}_{i} , {y}_{i + j} 〉 \approx \frac{1}{N - j} {\sum }_{i = 1}^{N - j} {y}_{i} {y}_{i + j} . \qquad ( 7 )$$

For the case of interest in predicting the next data value from a set of evenly-spaced data, the estimation equation [Equation (2)] takes the form

$${y}_{n} = {\sum }_{j = 1}^{M} {d}_{j} {y}_{n - j} + {x}_{n} \qquad ( 8 )$$

and Equation (5) becomes the set of M equations for the M unknown dⱼ's, which are now denoted the linear prediction (LP) coefficients,

$${\sum }_{j = 1}^{M} {φ}_{\vert j - k \vert} {d}_{j} = {φ}_{k} \\ ( k = 1 , K , M ) . \qquad ( 9 )$$

It should be noted that while noise does not appear explicitly in the three equations immediately above, it is properly accounted for provided it is point-to-point uncorrelated. “Noise” in this mathematical exposition refers to such noise terms as amplifier noise and not to the noise signal to be removed, such as the undesirable aircraft cabin sound field, which while is colloquially considered to be “noise” is in fact the signal for this problem. The aircraft cabin sound field does not necessarily have to be point-to-point uncorrelated and in fact it must not be for a linear predictive noise cancellation approach to be effective.

The mean discrepancy [symbol] xₙ² [symbol] may be estimated using Equation (7) as

[symbol]

xₙ² [symbol] =φ₀−φ₁d₁−φ₂d₂−Λ−φ_{M}d_{M}. (10)

To use these mathematical principles in a noise cancellation system, one must determine the MEM/linear prediction coefficients, the dⱼ's, using Equations (7) and (9), applied to a set of N data points, obtaining M coefficients. Then Equation (8) can be used to generate predictions of the sound field to be canceled, yₙ at timestep n. This prediction of the sound field is inverted by multiplication by −1, and the signal value −yₙ is sent to the speaker 116 after having been made into an analog signal by the digital-to-analog converter 114. At each time step, the expected discrepancy magnitude, |xₙ|, can be calculated from the square root of Equation (10) and when the expected discrepancy magnitude becomes unacceptably large, a new set of dⱼ's can be computed using the latest available set of yₙ's.

Alternatively, the MEM/linear prediction coefficients can be re-generated on a fixed schedule, that is, after a preset number of new data points are collected. In the preferred embodiment of this invention, when sufficient computational resources are available, a new set of MEM/linear prediction coefficients are computed after each new sound field data point is collected, and extrapolation to obtain a new yₙ is made over only a single sampling interval. This ensures the most accurate extrapolation to a cancellation value for the noise field for a given set of values of M and N. This is desirable because the computational requirements of the approach of the invention are proportional to MN and by extrapolating by only a single data point, the computational requirements can be minimized for any given desired engineering tolerance specification for the accuracy of the noise cancellation.

Estimation of the noise signal to be cancelled, as in Equation (8), can be viewed as a generalization of a digital filter in which the MEM/linear prediction coefficients, the dⱼ's, are viewed as the filter coefficients. From such a viewpoint, the stability of the filtering process can be viewed in general systems theory by analysis of the roots of the characteristic polynomial

$${z}^{N} - {\sum }_{j = 1}^{N} {d}_{j} {z}^{N - j} = 0. \qquad ( 11 )$$

Stability requires that all N complex roots of the characteristic polynomial lie within the unit circle, i.e.,

|z|≦1. (12)

In general, there is no guarantee that the roots of the characteristic polynomial will lie within the unit circle, particularly in the presence of noise. Various prescriptions can be adopted for “massaging” the roots of the characteristic polynomial of the MEM/linear prediction coefficients in the event that stability problems in the noise cancellation arise, for example if root zᵢ is outside the unit circle, it can be mapped onto the unit circle by

$${z}_{i} -> \frac{{z}_{i}}{\vert {z}_{i} \vert} , \qquad ( 13 )$$

or alternatively, a root can be mapped across the unit circle by the prescription,

$${z}_{i} -> \frac{1}{{z}_{i}^{*}} , \qquad ( 14 )$$

that is, the reciprocal of the complex conjugate of the complex root. Any of these schemes for improving stability makes an implicit assumption about the character of the noise process to be cancelled. If roots outside the unit circle are to be permitted, this corresponds to the assumption that the noise signal to be cancelled is a superposition of exponentially growing as well as exponentially decaying complex sinusoids. As observed above, this corresponds well to the description of aircraft cabin noise and so that is the preferred embodiment. The prescriptions of Equations (13) and (14) as well as others that may occur to workers skilled in the art are taken to fall within the scope of this invention.

FIGS. 8A, 8B and 8C illustrate results from the application of the MEM noise cancellation approach of the invention to recorded aircraft cabin noise data. FIG. 8A illustrates 500 samples of recorded aircraft noise data 132. This data was collected at a sample rate of 11025 samples per second. The vertical scale is arbitrary, but consistent with FIGS. 8B and 8C. FIG. 8B illustrates the resulting trace 138 from the same data after the MEM noise cancellation approach of the invention has been applied using a fit of three MEM coefficients over a data window of ten samples. It should be noted that the trace scale has been expanded by a factor of four so as to show some structure in the MEM noise cancelled trace 138. A further application of the MEM noise cancellation approach of the invention using a data window of twenty points and ten MEM coefficients is illustrated in FIG. 8C, trace 140. The vertical scale is the same as for the trace 138, but it is clear that still more noise has been cancelled. FIGS. 8A, 8B and 8C illustrate that very good noise cancellation can be achieved using the predictive approach based on MEM, even while using a relatively modest number of coefficients and a correspondingly small amount of computation.

While the present invention has been particularly shown and described with reference to exemplary embodiments thereof, it will be understood by those of ordinary skill in the art that various changes in form and details may be made therein without departing from the spirit and scope of the present invention as defined by the following claims.

1. A method of active noise cancellation in a volume comprising:
generating a mathematical model of a noise process to be cancelled;
constructing an extrapolated noise signal from the mathematical model;
inverting the extrapolated signal;
applying the inverted signal to the volume; and
canceling the noise in the volume using the applied signal.

2. The method of claim 1, further comprising:
measuring the noise signal close to the volume;
converting the noise signal to a digital signal; and
inputting the noise signal to the mathematical model.

3. The method of claim 1, further comprising, before applying the inverted signal to the volume, converting the inverted signal into an analog signal.

4. The method of claim 1, further comprising generating an anti-noise sound wave by a speaker operating within the volume.

5. The method of claim 4, wherein destructive interference of the noise signal and the anti-noise sound wave cancels the noise.

6. The method of claim 1, wherein the mathematical model is generated using at least one of linear predictive coding and maximum entropy method.

7. The method of claim 1, wherein the mathematical model is used to predict the noise signal in the next sample.

8. The method of claim 7, wherein the predicted signal for the next sample is used to cancel the noise signal in the next sample.

9. The method of claim 1, wherein the mathematical model is periodically updated.

10. The method of claim 1, wherein canceling the noise comprises canceling tonal and non-tonal noise components.

11. The method of claim 1, further comprising measuring the noise outside the volume.

12. The method of claim 1, further comprising measuring the noise outside the volume and measuring the noise within the volume.

13. The method of claim 1, wherein canceling the noise comprises canceling periodic noise and noise generated by turbulent air flow.

14. The method of claim 1, wherein the volume comprises at least one of a helmet, a headset, and speakers close to a head.

15. A method of active noise cancellation in a volume comprising:
measuring a noise signal;
predicting a noise signal in a next sample period from the measured noise signal;
inverting the predicted signal;
applying the inverted signal to the volume; and
canceling the noise signal in the next sample period using the predicted signal.

16. The method of claim 15, further comprising, prior to predicting the noise signal, converting the noise signal to a digital signal.

17. The method of claim 15, further comprising, before applying the inverted signal to the volume, converting the inverted signal into an analog signal.

18. The method of claim 15, further comprising generating an anti-noise sound wave by a speaker operating within the volume.

19. The method of claim 18, wherein destructive interference of the noise signal and the anti-noise sound wave cancels the noise.

20. The method of claim 15, wherein the predicted noise signal is generated using at least one of linear predictive coding and a maximum entropy method.

21. The method of claim 15, wherein canceling the noise signal comprises canceling tonal and non-tonal noise components.

22. The method of claim 15, further comprising measuring the noise outside the volume.

23. The method of claim 15, further comprising measuring the noise outside the volume and measuring the noise within the volume.

24. The method of claim 15, wherein canceling the noise signal comprises canceling periodic noise and noise generated by turbulent air flow.

25. The method of claim 15, wherein the volume comprises at least one of a helmet, a headset, and speakers close to a head.

26. An apparatus for active noise cancellation in a volume comprising:
a first microphone that senses noise;
a noise model generator that receives the sensed noise signal and generates a mathematical model of a noise process to be cancelled;
an extrapolator that extrapolates a noise signal from the mathematical model; and
an inverter that inverts the extrapolated signal and applies the signal to a volume.

27. The apparatus of claim 26, further comprising an analog-to-digital converter that receives the sensed noise signal from the first microphone and applies a digitized equivalent of an analog signal to the noise model generator.

28. The apparatus of claim 26, further comprising a digital-to-analog converter that receives the inverted signal from the inverter and applies an equivalent analog form of the digital signal to the volume.

29. The apparatus of claim 26, wherein the first microphone senses the noise outside the volume.

30. The apparatus of claim 26, further comprising a second microphone.

31. The apparatus of claim 30, wherein the second microphone senses the noise signal within the volume.

32. The apparatus of claim 26, wherein the noise model generator comprises at least one of a digital signal processor, a computer and a field-programmable gate array.

33. The apparatus of claim 26, wherein the noise model generator uses at least one of linear predictive coding and a maximum entropy method.

34. The apparatus of claim 26, wherein the noise model generator calculates an extrapolated value of the noise signal for a next sample period.

35. The apparatus of claim 26, wherein the extrapolator constructs the extrapolated noise signal for a future sample period.

36. The apparatus of claim 26, wherein the mathematical model is periodically updated.

37. The apparatus of claim 26, wherein the apparatus cancels tonal and non-tonal noise components.

38. The apparatus of claim 26, wherein the apparatus cancels periodic noise and noise generated by turbulent air flow.

39. The apparatus of claim 26, wherein the volume comprises at least one of a helmet, a headset, and speakers close to a head.

40. An apparatus for active noise cancellation in a volume comprising:
a first microphone that senses a noise signal;
a noise predictor that predicts a noise signal in a next sample period from the sensed noise signal;
an extrapolator that extrapolates a noise signal from the predicted noise signal; and
an inverter that inverts the extrapolated signal and applies the inverted signal to the volume.

41. The apparatus of claim 40, further comprising an analog-to-digital converter that receives the sensed noise signal from the first microphone and applies a digitized equivalent of an analog signal to the noise predictor.

42. The apparatus of claim 40, further comprising a digital-to-analog converter that receives the inverted signal from the inverter and applies an equivalent analog form of the digital signal to the volume.

43. The apparatus of claim 40, wherein the first microphone senses the noise outside the volume.

44. The apparatus of claim 40, further comprising a second microphone.

45. The apparatus of claim 44, wherein the second microphone senses the noise signal within the volume.

46. The apparatus of claim 40, wherein the noise predictor comprises at least one of a digital signal processor, a computer and a field-programmable gate array.

47. The apparatus of claim 40, wherein the noise predictor uses at least one of linear predictive coding and maximum entropy method.

48. The apparatus of claim 40, wherein the noise predictor calculates an extrapolated value of the noise signal for a next sample period.

49. The apparatus of claim 40, wherein the extrapolator constructs the extrapolated noise signal for a signal for a next sample period.

50. The apparatus of claim 40, wherein the predicted noise signal is periodically updated.

51. The apparatus of claim 40, wherein the apparatus cancels tonal and non-tonal noise components.

52. The apparatus of claim 40, wherein the apparatus cancels periodic noise and noise generated by turbulent air flow.

53. The apparatus of claim 40, wherein the volume comprises at least one of a helmet, a headset, and speakers close to a head.
